(19) United States
(12) Patent Application Publication
(10) Pub. No.: US 2026/0038656 A1
(43) Pub. Date: Feb. 5, 2026

(54) Artificial-Intelligence-Based Closed-Loop Information System and Data Validation

(71) Applicant: Aingelus Custos, Inc., Taylorsville, KY (US)

(72) Inventors: James E. Smith, Taylorsville, KY (US); Marcin B. Bauza, Allentown, PA (US); Jakub Misiorny, London (GB)

(21) Appl. No.: 19/288,841

(22) Filed: Aug. 1, 2025

Related U.S. Application Data
(60) Provisional application No. 63/679,075, filed on Aug. 2, 2024.

(51) Int. Cl. G16H 10/60; G06F 9/451

(52) U.S. Cl. CPC G16H 10/60; G06F 9/451

(57) ABSTRACT

A system includes memory hardware configured to store instructions and processor hardware configured to execute the instructions stored by the memory hardware. The instructions include retrieving a set of protocol data and retrieving a set of user data associated with an individual. The set of user data includes historical data and current data associated with the individual. The instructions include determining, a set of standards and comparing the set of standards and the set of user data. The instructions include, in response to a determination that the set of standards has been met, presenting an indication with a first formatting. The instructions include, in response to a determination that one or more standards of the set of standards has not been met, presenting a second indication with a second formatting.

### CROSS-REFERENCE TO RELATED APPLICATIONS

This application claims the benefit of U.S. Provisional Application No. 63/679,075 filed Aug. 2, 2024. The entire disclosure of the above application is incorporated by reference.

### FIELD

The present disclosure relates to automated data validation and, more particularly, to machine learning systems for validation of gathered data.

### BACKGROUND

Before the digital era, healthcare providers benefited from streamlined and efficient system centered around a physical bedside chart. This chart provided immediate access to all pertinent patient data in one place, allowing providers to make quick, informed decisions without needing to navigate multiple systems. The simplicity and accessibility of this method significantly enhanced productivity and responsiveness, especially in urgent clinical situations.

However, the healthcare landscape began to shift with the introduction of numerous digital systems, each housing different sets of critical patient information. This fragmentation forces providers to log into multiple platforms to retrieve and input data, even during emergencies. The time lost navigating these systems could delay life-saving decisions and highlights a growing inefficiency in modern healthcare data management.

Recording, updating, and validating user data is an essential operation in many applications. Incorrect data can result in errors which cause inefficiencies such as increased human oversight and processing time. The correction and verification of user data is time-intensive and can quickly scale beyond the human capability to manually correct. In some implementations, universal access to updated and valid data is critical to user safety. In a healthcare setting, the consequences of incorrect, difficult to understand, or inaccessible data can be life-threatening. For these reasons, a solution is needed to streamline healthcare data access to ensure accurate, easily accessible, and easily digestible data.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A system includes memory hardware configured to store instructions and processor hardware configured to execute instructions stored by the memory hardware. The instructions include retrieving a set of protocol data from a first set of data sources. The instructions include retrieving a set of user data associated with an individual. The set of user data includes historical data associated with the individual from a second set of data sources, and current data associated with the individual. The current data includes at least one of a set of real-time data from a set of sensors monitoring the individual, or a set of data from a threshold period of time. The instructions include determining, based on the set of protocol data, a set of standards. The instructions include comparing the set of standards and the set of user data to determine whether the set of standards has been met. The instructions include, in response to a determination that the set of standards has been met, presenting, via a user interface, an indication with a first formatting. The instructions include, in response to a determination that one or more standards of the set of standards has not been met, presenting, via the user interface, a second indication with a second formatting.

In other features, the first set of data sources includes at least one of a scholarly research database, a compliance metric database, a dietary database, an external alerting system, or an external guideline repository. In other features, the set of protocol data includes at least one of a clinical guideline, a regional alert, a national alert, or a government agency alert.

In other features, the threshold period of time is based on a current status of the individual, the threshold period of time is based on a current location of the individual, and the threshold period of time is contiguous with a current time.

In other features, the instructions include retrieving a second set of user data associated with a second individual, and comparing the set of standards, the set of user data, and the second set of user data to determine a priority of the individual and a priority of the second individual.

In other features, the first formatting and the second formatting include at least one of a first color and a second color, a first font and a second font, a first indicator and a second indicator, a first text size and a second text size, or a first alert and a second alert.

In other features, retrieving the set of user data is performed in response to a determination that a set of authorization criteria has been met, and in response to a determination that the set of authorization criteria has not be met, transmitting an authorization request to a device associated with the individual.

In other features, the instructions include presenting the historical data in a first manner, receiving a set of user feedback, and based on the set of user feedback, presenting the historical data in a second manner.

In other features, the instructions include receiving a set of pre-authorization data, and in response to a determination that a set of authorization criteria has been met, transmitting a data subset of the set of user data to a set of entities. The data subset is specified by the set of pre-authorization data. The set of entities is specified by the set of pre-authorization data. The set of authorization criteria is specified by the set of pre-authorization data. The set of authorization criteria includes a status of the individual. The set of pre-authorization data is generated based on a set of user inputs.

In other features, determining the set of standards is performed by a machine learning model. In other features, the historical data includes at least one of diagnostic records, treatment records, surgical notes, medication logs, information provided by the individual, wearable device telemetry, laboratory reports, radiology reports, or family genetic data. In other features, the second set of data sources includes a first healthcare provider and a second healthcare provider.

In other features, the system is a distributed computing system that includes one or more sets of remote processing hardware and one or more sets of remote memory.

In other features, the current data includes the set of real-time data from the set of sensors. In other features, the set of sensors is located in a first location. In other features, the memory hardware and the processor hardware are located in the first location.

In other features, the individual is located at a first location. The memory hardware and the processor hardware are located at the first location.

In other features, the instructions include determining a respective role of a user interacting with the user interface. The respective role is one of a set of enumerated roles. The set of enumerated roles includes a first role and a second role. In other features, the instructions include, in response to determining that the respective role is the first role, generating the user interface with a first set of elements. In other features, the instructions include, in response to determining that the respective role is the second role, generating the user interface with a second set of elements.

A method includes retrieving a set of protocol data from a first set of data sources. The method includes retrieving a set of user data associated with an individual. The set of user data includes historical data associated with the individual from a second set of data sources, and current data associated with the individual. The current data includes at least one of a set of real-time data from a set of sensors monitoring the individual, or a set of data from a threshold period of time. The method includes determining, based on the set of protocol data, a set of standards. The method includes comparing the set of standards and the set of user data to determine whether the set of standards has been met. The method includes, in response to a determination that the set of standards has been met, presenting, via a user interface, an indication with a first formatting. The method includes, in response to a determination that one or more standards of the set of standards has not been met, presenting, via the user interface, a second indication with a second formatting.

In other features, the method includes retrieving a second set of user data associated with a second individual, and comparing the set of standards, the set of user data, and the second set of user data to determine a priority of the individual and a priority of the second individual.

In other features, the method includes receiving a set of pre-authorization data, and in response to a determination that a set of authorization criteria has been met, transmitting a data subset of the set of user data to a set of entities. The data subset is specified by the set of pre-authorization data. The set of entities is specified by the set of pre-authorization data. The set of authorization criteria is specified by the set of pre-authorization data. The set of authorization criteria includes a status of the individual. The set of pre-authorization data is generated based on a set of user inputs.

A non-transitory computer-readable storage medium includes processor-executable instructions. The instructions include retrieving a set of protocol data from a first set of data sources. The instructions include retrieving a set of user data associated with an individual. The set of user data includes historical data associated with the individual from a second set of data sources, and current data associated with the individual. The current data includes at least one of a set of real-time data from a set of sensors monitoring the individual, or a set of data from a threshold period of time. The instructions include determining, based on the set of protocol data, a set of standards. The instructions include comparing the set of standards and the set of user data to determine whether the set of standards has been met. The instructions include, in response to a determination that the set of standards has been met, presenting, via a user interface, an indication with a first formatting. The instructions include, in response to a determination that one or more standards of the set of standards has not been met, presenting, via the user interface, a second indication with a second formatting.

In other features, the instructions include retrieving a second set of user data associated with a second individual, and comparing the set of standards, the set of user data, and the second set of user data to determine a priority of the individual and a priority of the second individual.

In other features, the instructions include receiving a set of pre-authorization data, and in response to a determination that a set of authorization criteria has been met, transmitting a data subset of the set of user data to a set of entities. The data subset is specified by the set of pre-authorization data. The set of entities is specified by the set of pre-authorization data. The set of authorization criteria is specified by the set of pre-authorization data. The set of authorization criteria includes a status of the individual. The set of pre-authorization data is generated based on a set of user inputs.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIGS. 1A-1C are a block diagram of an example artificial intelligence-based closed-loop information system.

FIGS. 2A-2B are a block diagram of an example artificial intelligence and machine learning model processing system.

FIGS. 3A-3B are a block diagram of an example dynamic triage system.

FIGS. 4A-4B are a block diagram of an example user interface system.

In the drawings, reference numbers may be reused to identify similar and/or identical elements.

### DETAILED DESCRIPTION

### Introduction

The present disclosure includes systems and methods for dynamic triage. Unlike traditional triage, which ends after the initial assessment, the systems and methods of the present disclosure continuously monitor patients and compare their conditions in real-time. This allows providers to prioritize care dynamically when multiple patients are in critical condition and ensure that the most urgent needs are addressed immediately.

The systems and methods of the present disclosure also enhance clinical decision-making by offering real-time recommendations and, in some cases, making decisions autonomously. The systems and methods include user-friendly graphic interfaces that simplify complex medical data, presenting only the most relevant information in a clear, visual format. The systems and methods of the present disclosure provide instant access to curated data across various devices, improving care coordination among providers, patients, and families alike.

### Dynamic Triage

The system presents a unified, real-time visual interface designed to optimize clinical decision-making through an intuitive and standardized layout. Upon system initialization, the interface displays a consolidated dashboard representing patients under a provider's care. Each patient is visually encoded using a simplified, system-compliant identifier, ensuring both clarity and interoperability with existing electronic health record (EHR) standards.

Central to the interface is a color-coded triage system that conveys patient status at a glance:
Green: Indicates that all monitored parameters are within expected ranges. No immediate action is required.
Yellow: Signals that attention will soon be required. This may include emerging trends, incomplete documentation, or upcoming clinical decision points.
Red: Denotes urgent or conflicting issues that require immediate provider intervention.

The color-coding is not static. Color-coding is dynamically generated by a backend analytics engine that continuously ingests and evaluates structured and unstructured data from multiple clinical systems. The system architecture supports real-time data ingestion from laboratory systems, radiology reports, physician notes, national guideline repositories, and external alerting systems (such as FDA safety notifications). In various embodiments, additional or alternative formatting is used such as animations, flashing indicators, alerts, pop-ups, bolding, underlining, etc. In various embodiments, alternative colors, fonts, text size, symbols, etc. are used.

Upon user interaction (specifically, the selection of a patient node within the interface) the system expands the visual element to display a detailed, context-specific explanation of the assigned color status.

For example, a red status may be triggered by a missed diagnostic test, a discrepancy between imaging results and clinical documentation, a newly identified drug interaction based on real-time pharmacovigilance data (for example, via real-time FDA reports), or a surgical site mismatch detected through cross-referencing procedural notes.

As another example, a yellow status may result from a lab value trending toward a critical threshold, a change in national clinical guidelines rendering current documentation outdated, a missed immunization, or exposure to an emerging regional health threat.

The system supports dynamic triage, a continuous evaluation model where patient prioritization is recalculated in real-time. When multiple patients are flagged red, the system prioritizes the patients based on urgency and projected clinical trajectory, enabling providers to allocate attention and resources efficiently under time-sensitive conditions.

The interface is designed for deployment across multiple access points, including bedside terminals, mobile devices, central command stations, and patient-facing portals. This ensures consistent access to the same visual and analytical framework across all stakeholders.

In critical scenarios, the system is capable of generating real-time clinical recommendations and, where authorized, executing automated pre-approved actions to mitigate risk during high-pressure events.

The visual layout, combined with the intelligent color-coded system and continuous triage logic, restores the immediacy and clarity of traditional bedside charts while leveraging modern data integration and decision-support technologies.

### Data Analysis

In various embodiments, the system integrates various data sources. For example, the system receives and analyzes data from diagnostic and treatment records, nurse charting, surgical notes, medication administration logs, patient-provided histories, wearable devices (such as heart rate monitors, blood pressure monitors, respiratory rate monitors, blood oxygenation monitors, thermometers, commercial smart watches, and/or other sensors), telemetry, family genetic data, and real-time updates from agencies like the Center for Disease Control and Prevention (CDC) and/or the Food and Drug Administration (FDA). In various embodiments, the system also integrates scholarly research, compliance metrics, environmental exposure data, and dietary databases. In various embodiments, data is analyzed and verified as it received. For example, if the patient's wearable device reports an irregular heartbeat and the nurse hasn't documented a concern, the system flags that discrepancy. As another example, if an order for a medication contraindicates with a genomic red flag in the patient's family health history, the system generates an alert (or changes the formatting or color-coding associated with user interface element associated with the patient). As another example, if a child shows signs that match a new illness pattern being reported in another region, the system notifies the provider and simultaneously alerts the CDC with permissioned metadata.

### User Interfaces

The system employs a dynamic graphical user interface (GUI) designed to optimize clinical decision-making through real-time data visualization and intelligent prioritization. The primary interface presents a continuously updating dashboard that aggregates patient data under a provider's care. This interface is dynamically refreshed and supports interactive operations such as scrolling, filtering, and searching. The system algorithmically prioritizes patients based on urgency, time-sensitive clinical thresholds, and predictive modeling of clinical trajectories.

Upon selection of a patient, the interface transitions to a context-sensitive display that renders only clinically relevant data. This relevance is determined by a combination of the patient's current condition and the clinical context. For example, in the case of a diabetic patient presenting with chest pain, the system automatically surfaces pertinent cardiovascular and metabolic data, including historical trends, lab results, and relevant literature on comorbid presentations. Irrelevant historical data is suppressed unless explicitly requested by the provider. The system integrates real-time clinical decision support by identifying significant findings and, when applicable, generating evidence-based recommendations derived from continuously updated medical literature and guidelines.

For pre-operative scenarios, the system executes a protocol-driven workflow engine that verifies procedural readiness. It confirms the completion of required diagnostics, consent documentation, and imaging consistency. Discrepancies or omissions are flagged, and the system provides direct access to the source documents. Providers can interact with these alerts through a single-click interface that supports annotation, verification, and escalation.

The system architecture is designed to augment, not replace, clinical judgment by offloading cognitive tasks such as cross-referencing disparate data sources (such as records from multiple healthcare providers, past healthcare records, health records from a current health care visit), resolving data inconsistencies, tracking evolving clinical guidelines, and monitoring compliance with time-sensitive protocols. These functions are executed in the background using a combination of machine learning algorithms, natural language processing (NLP), a longitudinal knowledge graph, and/or other types of algorithms and methods. This knowledge graph maintains temporal and semantic relationships across all patient data points, enabling the system to contextualize current findings within historical and predictive frameworks. In various embodiments, historical healthcare data (data from previous health care sessions from multiple providers and locations) and current healthcare data (for example, data from a current ongoing health care session or visit at a particular location) are combined.

In various embodiments, the system incorporates adaptive learning mechanisms. If the system detects patterns, such as repeated alert dismissal, the system modifies future alerts to reduce cognitive fatigue while preserving clinical safety. In various embodiments, the system requests and receives ratings of the utility of alerts, and these ratings are incorporated into the system's reinforcement learning loop to refine future outputs.

In various embodiments, the system generates a user interface for use by administrative users. Instead of patient-level data, this interface aggregates population-level trends. For instance, if the system detects an increase in critical alerts related to a specific pathology within a department, the interface highlights the trend and notifies relevant stakeholders. This enables immediate deployment of targeted interventions, such as staff re-education, based on the root cause identified by the system.

By enabling real-time detection and response to clinical and operational anomalies, the system reduces reliance on retrospective audits and post-incident reviews. The system is engineered to intercept errors before they result in patient harm, thereby transforming the safety and efficiency of clinical workflows.

### Compliance

In various embodiments, the system performs continuous, automated analysis of clinical documentation for billing accuracy and compliance. The system continuously monitors documentation for billing accuracy across all disease processes, diagnostics, and treatment plans. The system checks each case against performance metrics, ensuring that the steps required for optimal reimbursement are accurately documented. The system flags inconsistencies in real-time, so that issues can be addressed before claims are submitted.

In various embodiments, the system also includes a predictive analytics module for the prevention of adverse clinical events commonly referred to as “never events,” such as wrong-site surgeries, retained surgical items, and drug contraindications. The system continuously monitors procedural workflows and data inputs for deviations from established protocols. When a required step is omitted or an order conflicts with standard practice, the system generates an immediate alert. This alerting mechanism is designed to allow clinical staff to intervene before the event occurs, thereby mitigating risk and enhancing both patient safety and institutional financial integrity.

In various embodiments, the system automates administrative oversight functions traditionally performed through manual chart review. In various embodiments, the system conducts real-time cross-referencing of clinical documentation against regulatory indicators, such as those defined by the Centers for Medicare & Medicaid Services (CMS). In various embodiments, the system maintains a comprehensive audit trail that records all alerts, user interactions, and resolutions, including timestamps and user identifiers. This audit functionality supports accreditation and compliance reporting by providing a verifiable timeline of system activity and user response. By eliminating the need for retrospective investigation, the system enhances operational efficiency and ensures that administrators have immediate access to actionable information to prevent errors before they occur.

### Efficiency

For providers, one of the most meaningful benefits is the reduction in screen time. The system drastically cuts down the number of systems a provider needs to access, which gives more face time with patients, more time seeing additional patients, and most importantly, better work-life balance. The burden of staring into a screen for hours, piecing together incomplete and often contradictory information, is gone.

### Patient User Interface

In various embodiments, the system includes a patient user interface that connects to the interface used by healthcare providers. The system implements a decentralized data access protocol that allows patients to retain exclusive control over their health information. When a healthcare provider requires access to a specific medical record, the system initiates a secure, asynchronous request to the patient using a configurable communication channel. Supported channels include SMS, email, push notifications, and encrypted in-app alerts. The request includes metadata such as the requesting entity, data scope, and purpose, and includes user interface elements for authorizing or denying access through a secure interface.

Once patient authorization has been received, the system executes a secure data transmission using end-to-end encryption protocols, ensuring immediate availability of the requested records. For patients that are not yet enrolled, the system supports dynamic onboarding via the same communication channels, enabling real-time account creation and interface provisioning. The patient interface is dynamically rendered based on user-specific comprehension metrics. In various embodiments, the system uses natural language processing to simplify medical terminology and present data in a visual, context-aware format.

In various embodiments, the system employs adaptive learning algorithms and natural language processing to optimize patient communication. In various embodiments, the system monitors interaction patterns and linguistic comprehension, adjusting content delivery accordingly. This includes rephrasing clinical explanations, modifying interface layouts, and tailoring recommendations to the patient's cognitive profile. In various embodiments, these optimizations and adaptations are based on user feedback, recorded communications (written or auditory) between a user and healthcare provider, and/or health care notes about patient comprehensive levels.

In various embodiments, the system includes a natural language interface that allows patients to input symptoms and receive algorithmically generated care recommendations. These may include home monitoring protocols, scheduled provider visits, or escalation to emergency services. For inquiries regarding diagnoses or treatment plans, the system can either provide direct responses based on its knowledge graph or act as a secure intermediary to communicate with the provider. Upon patient consent, the system transmits the query, retrieves the provider's response, and reformats it into simplified language for patient comprehension. This bidirectional communication framework enhances patient autonomy, improves care coordination, and ensures clarity across all stages of the healthcare journey.

In various embodiments, the system incorporates a pre-authorization framework for emergency scenarios. Using the system, patients designate specific providers or healthcare systems with conditional access rights to their records in the event of incapacitation. This access is governed by predefined clinical relevance filters and is logged for auditability. In various embodiments, in emergency contexts, the system automatically validates the provider's credentials and delivers a curated dataset optimized for immediate clinical decision-making. If additional data is requested beyond the pre-authorized scope, the system alerts users, queues the request, and awaits patient or legally designated proxy consent.

In various embodiments, the system supports cross-network interoperability (for example, In geographically remote or travel-related emergencies). Using the system, a local provider can initiate a data access request, which is processed through the same secure authorization workflow. Upon approval, the system generates a situationally relevant summary of the patient's medical history, including diagnostics, medications, allergies, and recent treatments. Additional data can be requested and transmitted in real-time, independent of the patient's physical location.

### System Architecture

In various embodiments, the system operates as an independent system between existing healthcare systems and end users. The system maintains its own data processing infrastructure, storage systems, and computation engines. The system creates a unified data model by ingesting, processing, and storing data from disparate healthcare systems. The system stores intermediate processing results, algorithmic reasoning chains, and derived intelligence products. The system provides deep-linking capabilities back to source systems while maintaining its own processed data layer.

In various embodiments, the system uses multi-source data integration. In various embodiments, the system includes i) real-time data ingestion through multiple connection methods such as REST/SOAP APIs, direct database connections, HL7 interfaces, FHIR protocols, etc.; ii) automated data synchronization engines that continuously monitor source systems for updates; iii) data transformation pipelines that normalize disparate data formats into unified schemas; iv) conflict resolution algorithms that reconcile inconsistencies across multiple data sources; and v) timestamp-based data versioning to track changes and maintain audit trails.

In various embodiments, one or more elements of the system (such as modules, storage, memory, or processing hardware) is cloud-based or distributed. In other words, one or more elements of the system is remote from one or more of the users (such as a patient, nurse, doctor, and/or administrator) or other elements of the system. In various embodiments, the system is located on the premises of a healthcare provider (such as hospital), the same location as a patient, and/or the set of sensors or wearable devices monitoring the patients.

### Artificial Intelligence and Machine Learning

In various embodiments, the system includes various machine learning models. In various embodiments, the system includes a natural language processing engine. In various embodiments, the natural language processing engine includes i) LLM-powered parsing of unstructured clinical notes, reports, and documentation; ii) named entity recognition for medical terminology, drug names, dosages, and clinical indicators; iii) sentiment analysis and urgency detection in clinical documentation; iv) automated structuring of free-text entries into machine-readable formats; and v) context-aware interpretation that considers medical domain knowledge.

In various embodiments, the system includes a pattern recognition and prediction module. In various embodiments, the pattern recognition and prediction module i) includes supervised learning models trained on historical patient outcomes and clinical trajectories; ii) includes unsupervised clustering algorithms to identify patient cohorts and risk patterns; iii) performs time-series forecasting for patient deterioration prediction and resource planning; iv) includes anomaly detection algorithms that flag unusual patient status changes or data inconsistencies; and v) uses ensemble methods combining multiple ML models for robust decision-making.

In various embodiments, the system includes generative artificial intelligence models. In various embodiments, the system includes LLMs for generating clinical summaries, care recommendations, and patient explanations. In various embodiments, the system performs automated rule generation from high-level clinical guidelines input by hospital staff and gathered from evidence-based practice resources. In various embodiments, the system performs dynamic content generation tailored to user roles and patient contexts. In various embodiments, the system performs real-time explanation generation for system recommendations and alerts.

### Dynamic User Interface System

In various embodiments, the system includes a context-aware rendering engine. In various embodiments, the context aware-rendering engine includes i) multi-factor UI adaptation based on various factors such as user role (such as patient, nurse, doctor, or administrator), patient acuity level, clinical context, device type, and time constraints; ii) rule-based logic trees combined with ML models to determine optimal information presentation; iii) real-time UI reconfiguration as patient status or clinical context changes; and iv) hierarchical information architecture that surfaces the most critical data first.

In various embodiments, the system generates a device-responsive interface. In various embodiments, the device responsive interface includes i) a web-based application with device-specific routing and rendering modes; ii) responsive design frameworks with breakpoints optimized for various devices such as bedside tablets, mobile devices, desktop workstations, and/or wall-mounted displays; iii) touch-optimized interfaces for bedside use and keyboard/mouse optimization for administrative tasks; and iv) offline capability for critical functions during network disruptions.

### Intelligent Rule Engine and Decision Support

In various embodiments, the system includes a hybrid rule processing system. In various embodiments, the hybrid rule processing system includes an in-house rule engine that processes both pre-programmed clinical protocols and dynamically generated rules. In various embodiments, the hybrid rule processing system includes ML-powered rule generation from high-level clinical guidelines provided by hospital staff and obtained from evidence-based practice resources. In various embodiments, the hybrid rule processing system includes LLM integration which interprets complex clinical scenarios and applies appropriate rule sets. In various embodiments, the hybrid rule processing system includes configurable rule hierarchies allowing hospital-specific customization while maintaining core safety protocols.

In various embodiments, the system includes a dynamic triage and prioritization module. In various embodiments, the dynamic triage and prioritization module includes continuous patient comparison algorithms that rank patients by clinical urgency and resource requirements. In various embodiments, the dynamic triage and prioritization module includes real-time risk stratification using multiple data streams (such as vitals, labs, imaging, and/or clinical notes). In various embodiments, the dynamic triage and prioritization module includes automated escalation protocols triggered by specific clinical combinations or deterioration patterns. In various embodiments, the dynamic triage and prioritization module includes load balancing algorithms that consider provider workload and patient acuity simultaneously.

### Real-Time External Data Integration

In various embodiments, the system includes a real-time external data integration module. In various embodiments, the real-time external data integration module i) automatically ingests data from the CDC, FDA, and other regulatory databases through APIs and data feeds; ii) web scrapes sources without formal APIs; iii) generates real-time alerts based on correlations between patient data and emerging public health warnings; and iv) determines geographic and temporal correlation of patient symptoms with regional health trends.

In various embodiments, the system includes an adaptive data acquisition module. In various embodiments, the adaptive data acquisition module
i) evaluates and selects sources based on data freshness, reliability, and relevance;
ii) determines fallback sources when primary data sources are unavailable;
iii) automatically verifies data through cross-referencing multiple sources; and
iv) caches and prioritizes systems for critical external data feeds.

### Clinical Protocol Monitoring and Compliance

In various embodiments, the system includes an automated protocol adherence tracking module. In various embodiments, the automated protocol adherence tracking module i) monitors clinical protocols (such as protocols for sepsis, stroke, cardiac care, etc.) through structured and unstructured data analysis in real-time; ii) compares timestamps of clinical interventions against protocol requirements; iii) deploys ap analysis algorithms that identify missed steps or delayed actions; and iv) triggers automated alerts and reminders that are integrated into hospital workflow systems.

In various embodiments, the system includes a documentation analysis module. In various embodiments, the documentation analysis module i) analyzes, via an LLM, clinical notes to extract structured protocol compliance data; ii) cross-references documentation against ordered tests, medications, and procedures; iii) automatically identifies documentation gaps that could affect reimbursement or compliance; iv) generates compliance reports with supporting evidence and recommendations.

### Data Security and Access Control

In various embodiments, the system includes a granular authorization framework module. In various embodiments, the granular authorization framework module i) oversees role-based access control with context-sensitive permissions; ii) oversees patient-controlled data sharing with granular consent management; iii) automatically generates audit trails for all data access and system interactions; iv) oversees emergency access protocols with post-hoc authorization and logging.

In various embodiments, the system includes a privacy preservation module. In various embodiments, the privacy preservation module i) executes de-identification algorithms for analytics while maintaining clinical utility; ii) performs secure multi-party computation for cross-institutional data sharing; iii) encrypts at rest and in transit all patient data and system communications; iv) oversees compliance frameworks for HIPAA, state privacy laws, and institutional policies.

These technical components work together to create a comprehensive healthcare intelligence platform that transforms fragmented healthcare data into actionable insights while maintaining security, compliance, and clinical workflow integration.

### Block Diagrams

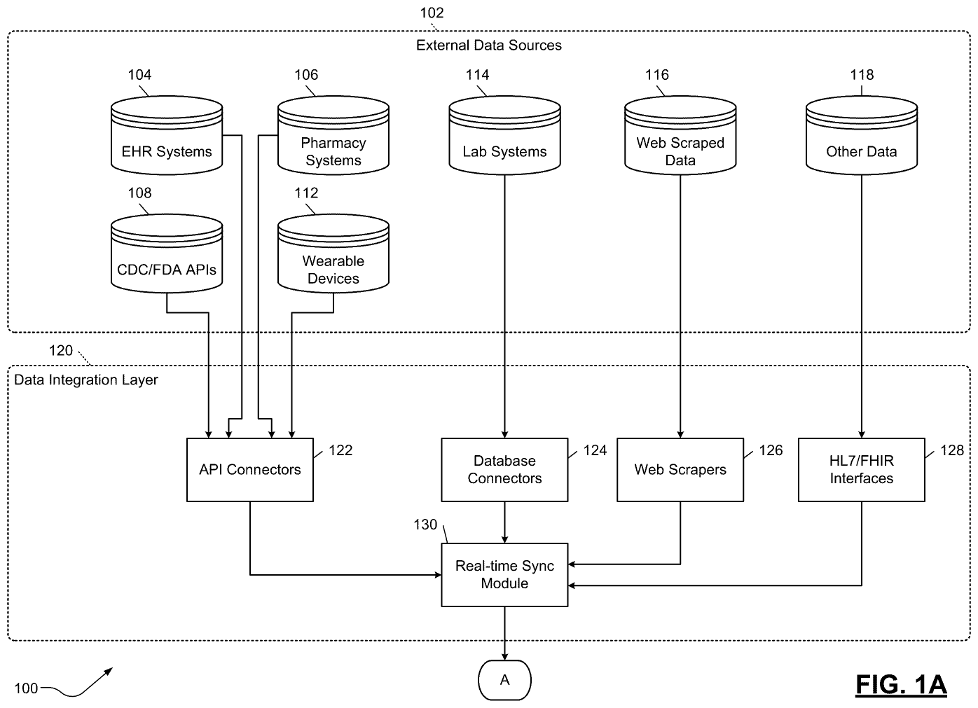

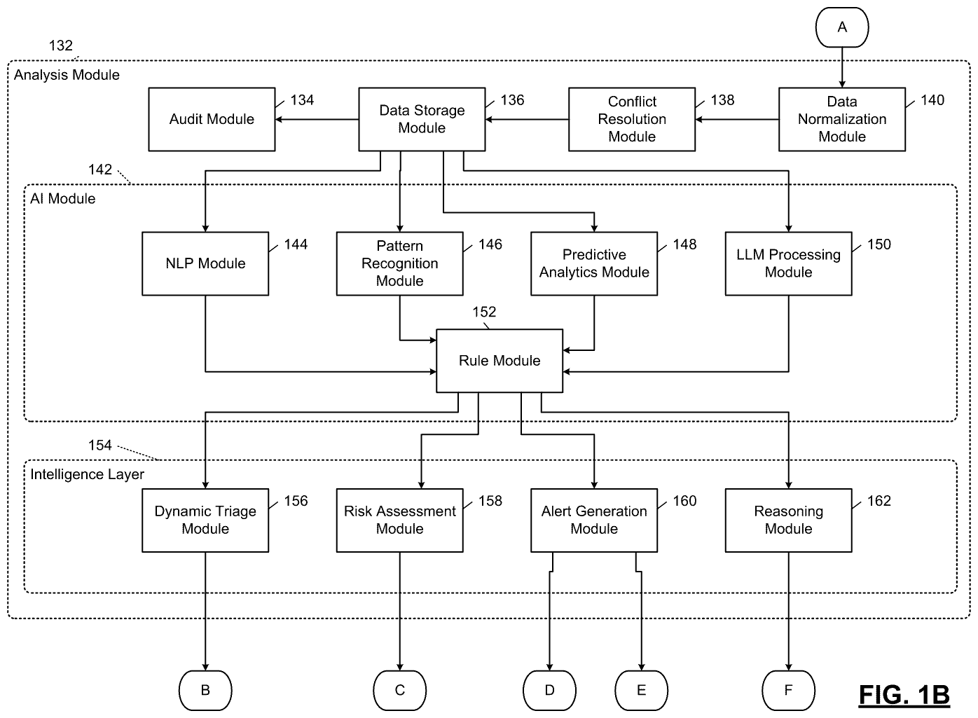

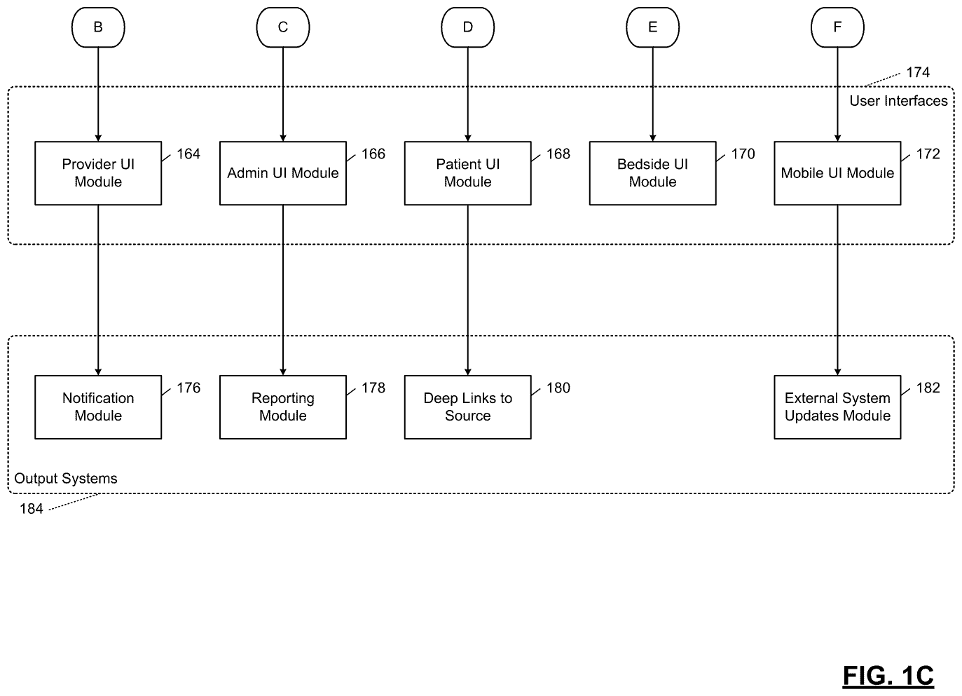

FIGS. 1A-1C are a block diagram of example closed loop information system 100. System 100 includes data integration layer 120, analysis module 132, user interfaces 174, and output systems 184. In various embodiments, system 100 includes various modules as described below.

System 100 collects data from external data sources 102, which includes EHR systems 104, pharmacy systems 106, CDC/FDA APIs 108, wearable devices 112, lab systems 114, web scraped data 116, and other data 118. Data from external data sources 102 is collected by (or received at) data integration layer 120.

Data integration layer 120 includes API connectors 122, database connectors 124, web scrapers 126, HL7/FHIR interfaces 128, and real-time sync module 130. API connectors 122 collects data from data sources with APIs, such as EHR systems 104, pharmacy systems 106, CDC/FDA APIs 108, and wearable devices 112. Database connectors 124 collects data from various database sources, such as lab systems 114. Web scrapers 126 collects data from various unstructured online sources, which are represented by web scraped data 116. Other data 118 is collected by HL7/FHIR interfaces 128. Data collected by API connectors 122, database connectors 124, web scrapers 126, HL7/FHIR interfaces 128 is transmitted to real-time sync module 130.

Real-time sync module 130 transmits the data to analysis module 132, which includes AI module 142 and intelligence layer 154. The data from real-time sync module 130 is received by data normalization module 140 before being transmitted to conflict resolution module 138, data storage module 136, and audit module 134. Data storage module 136 transmits the data to AI module 142.

Data is transmitted to AI module 142, which includes various modules such as NLP module 144, pattern recognition module 146, predictive analytics module 148, and LLM processing module 150. Data processed by these modules is then processed by rule engine 152 to generate rules (and/or determine rule adherence). Rules from rule engine 152 are used by intelligence layer 154, which includes dynamic triage module 156, risk assessment module 158, alert generation module 160, and reasoning engine 162.

Data from intelligence layer 154 is used by user interfaces 174, which includes provider UI module 164, admin UI module 166, patient UI module 168, bedside UI module 170, and mobile UI module 172. Data from dynamic triage module 156 is used by provider UI module 164, data from risk assessment module 158 is used by admin UI module 166, data from alert generation module 160 is used by patient UI module 168 and bedside UI module 170, and data from reasoning engine 162 is used by mobile UI module 172. Data from user interfaces 174 is used by output systems 184, which includes notification module 176, reporting module 178, deep links to source 180, and external system updates module 182.

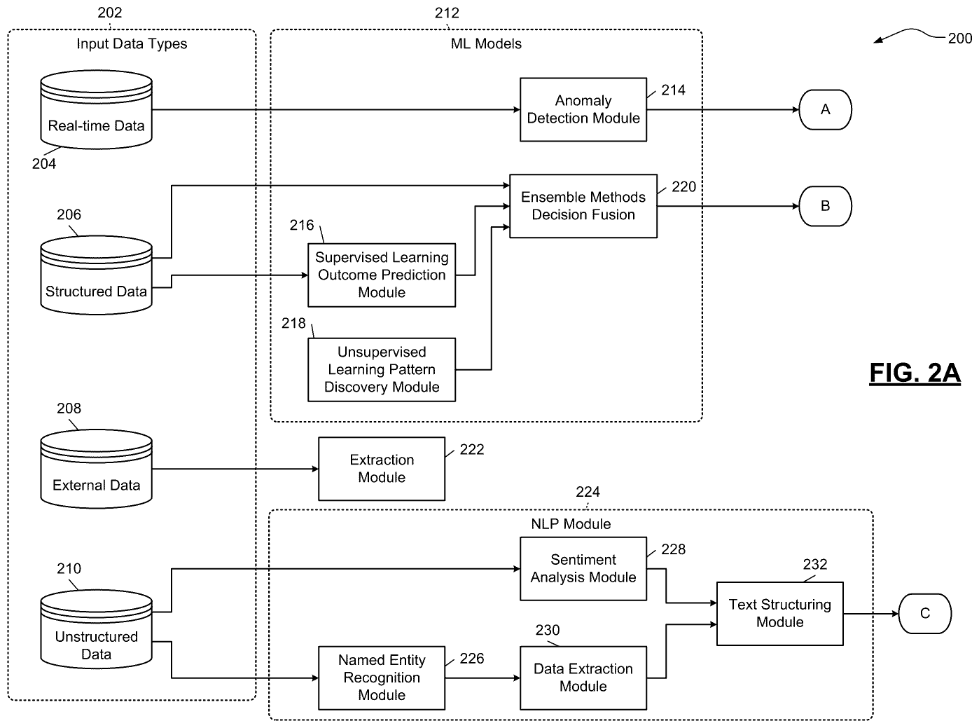

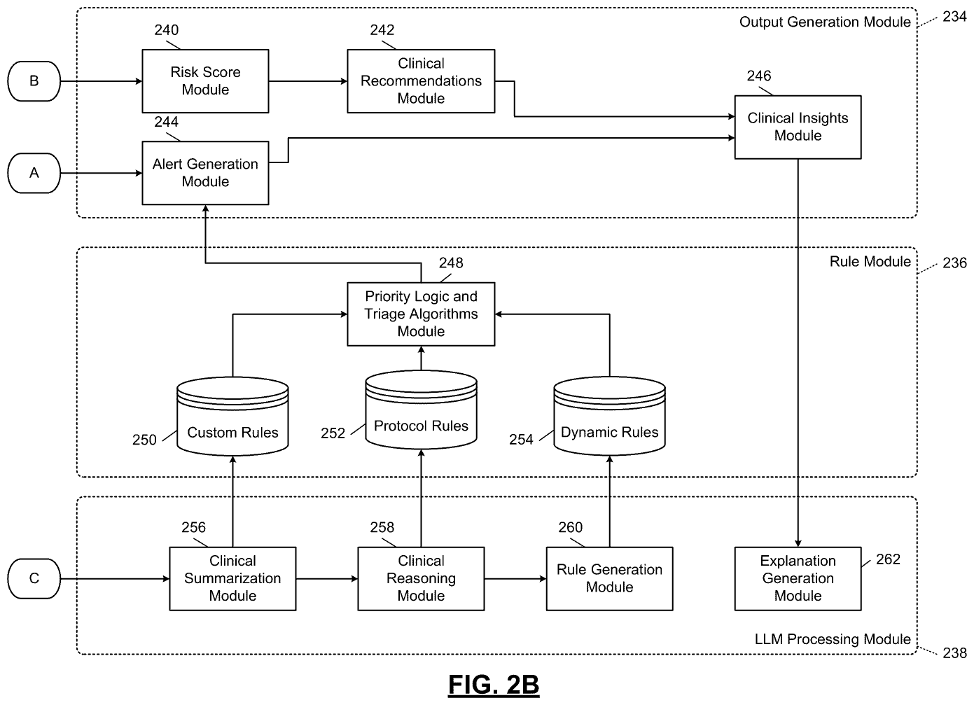

FIGS. 2A-2B are a block diagram of example artificial intelligence and machine learning model processing system 200, which in various embodiments is used by system 100. System 200 processes various input data types 202. Input data types 202 includes real-time data 204 (for example, streams from monitors and/or devices), structured data 206 (such as labs, vitals, and others), external data 208 (for example, data from the CDC, FDA, or other research repositories), and/or unstructured data 210 (for example, clinical notes and reports). System 200 uses machine learning models 212, natural language process (NLP) module 224, output generation module 234, rule module 236, and LLM processing module 238 to process input data types 202.

Real-time data 204 is processed by anomaly detection module 214 to identify data outliers. In various embodiments, if an anomaly is detected, alert generation module 244 generates an alert before transmitting the information to clinical insights module 246. In various embodiments, alert generation module 244 determines whether to generate an alert based on data from priority logic and triage algorithms module 248.

Structured data 206 is analyzed by supervised learning outcome prediction module 216 and ensemble methods decision fusion module 220. Ensemble methods decision fusion module 220 also receives predictions from unsupervised learning pattern discovery module 218. Structured data 206 and patterns from ensemble methods decision fusion module 220 is assigned a risk score by risk score module 240 before being analyzed by clinical recommendations module 242 and clinical insights module 246.

External data 208 is analyzed by extraction module 222. Unstructured data 210 by NLP module 224, which includes sentiment analysis module 228, named entity recognition module 226, data extraction module 230, and text structuring module 232. The analysis of NLP module 224 is then analyzed by LLM processing module 238, which includes clinical summarization module 256, clinical reasoning module 258, and rule generation module 260. The results of the clinical reasoning module 258 are stored protocol rules 252 rules. Rules from Rule generation module 260 are stored in dynamic rules 254.

The data (such as hospital-specific rules and/or requirements) from custom rules 250, protocol rules (such as rules for sepsis, stroke, etc.) from protocol rules 252, and dynamic rules 254 are used by priority logic and triage algorithms module 248 to triage and analyze the status of various individuals based on the status of real-time and historical data associated with the individual when compared to the various protocols, standards, and best practices. This information is passed through alert generation module 244, clinical insights module 246, and finally explanation generation module 262.

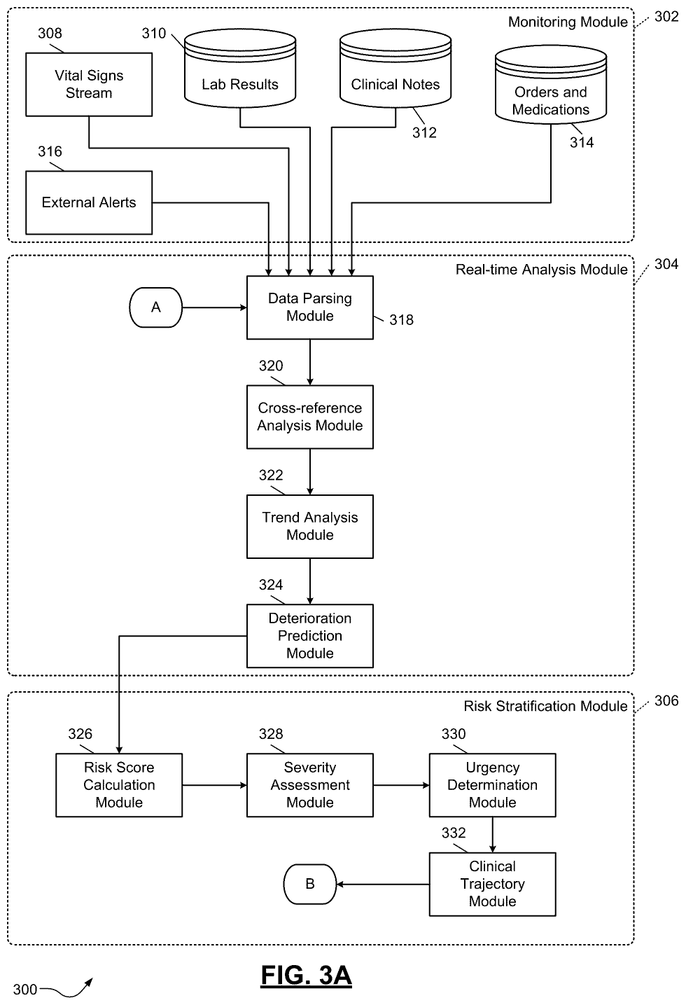

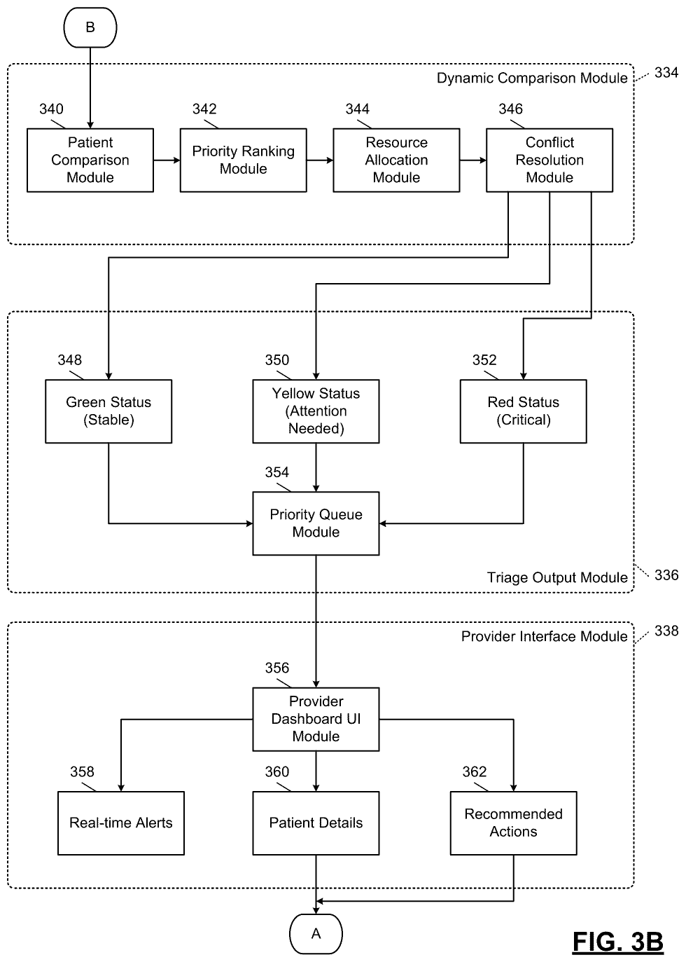

FIGS. 3A-3B are a block diagram of example dynamic triage system 300. System 300 includes monitoring module 302, real-time analysis module 304, risk stratification module 306, dynamic comparison module 334, triage output module 336, and provider interface module 338. Monitoring module 302 monitors data from vital signs stream 308, lab results 310, clinical notes 312, orders and medications 314, and external alerts 316. Once collected, the data is transmitted to real-time analysis module 304.

Within real-time analysis module 304, data is transmitted to data parsing module 318, cross-reference analysis module 320, trend analysis module 322, and deterioration prediction module 324. Data parsing module 318 also receives data from patient details 360 and recommended actions 362. Once analyzed, data from real-time analysis module 304 is transmitted to risk stratification module 306.

Risk stratification module 306 quantifies the risk associated with an individual or actions. Risk stratification module 306 includes risk score calculation module 326, severity assessment module 328, urgency determination module 330, and clinical trajectory module 332.

The data from risk stratification module 306 is analyzed by dynamic comparison module 334. Dynamic comparison module 334 compares the status of various individuals via patient comparison module 340, priority ranking module 342, resource allocation module 344, and confliction resolution module 346. Based on the data from dynamic comparison module 334, triage output module 336 generates a status for an individual, such as green 348 (stable), yellow 350 (attention needed), or red 352 (critical). The various individuals are then added to the priority queue managed by priority queue module 354 based on their status.

The priority queue is used by the provider interface module 338 to help healthcare providers to make quick decisions. Provider interface module 338 includes provider dashboard UI module 356, which includes real-time alerts 358, patient details 360, and recommended actions 362.

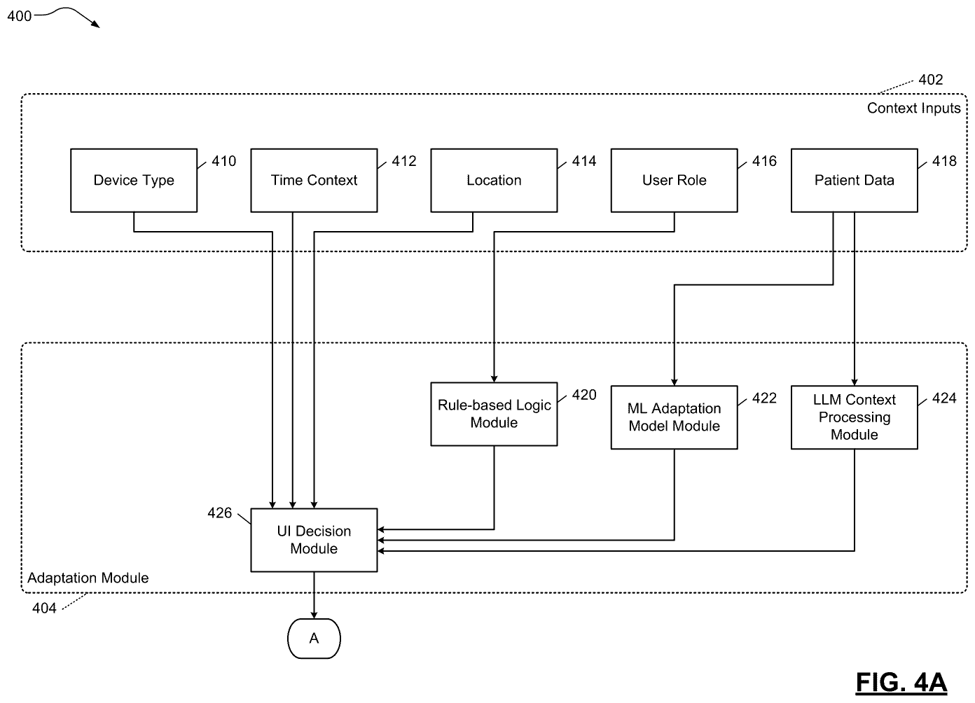

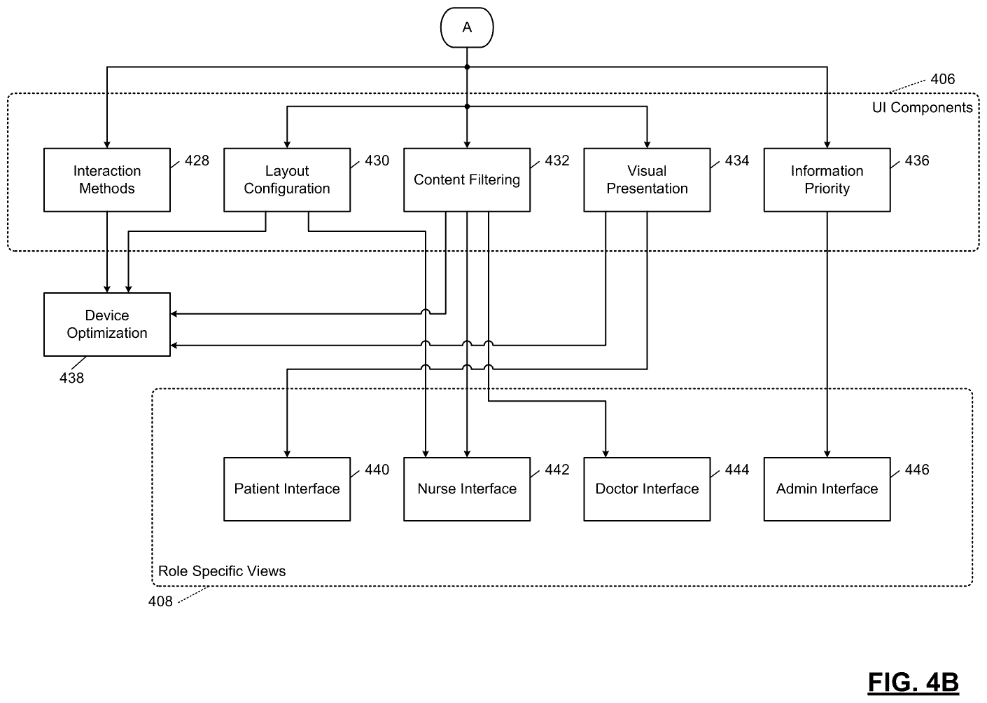

FIGS. 4A-4B are a block diagram of example user interface system 400. System 400 receives various context inputs 402. In various embodiments, context inputs 402 includes device type 410 (such as mobile, tablet, or desktop), time context 412 (such as shift, emergency, or routine), location 414 (such as bedside, station, or office), user role 416 (such as nurse, doctor, or admin), and patient data (such as stable, critical, emergency).

Context inputs 402 is used by adaptation module 404, which includes rule-based logic module 420, ML adaptation model module 422, LLM context processing module 424, and UI decision module 426. Rule-based logic module 420 receives data from user role 416. ML adaptation model module 422 and LLM context processing module 424 receive and analyze data from patient data 418.

UI decision module 426 receives the data from context inputs 402, rule-based logic module 420, ML adaptation model module 422, and LLM context processing module 424 to determine how the user interface should be presented and/or formatted.

Based on UI components 406, UI decision module 426 presents information to best fit the various devices and/or input methods for the various role specific views 408, which include patient interface 440, nurse interface 442, doctor interface 444, and admin interface 446. In various embodiments, UI components 406 (such as interaction methods 428, layout configuration 430, content filtering 432, visual presentation 434, and information priority 436) are optimized for each device and user role via device optimization 438.

### Key Features and Benefits

The method and system of the present disclosure include several key features and benefits. 1) Real-time health data integration that continuously monitors and updates a patient's health status using data from wearables and electronic health records. 2) Provider alerts that automatically alert healthcare providers to significant changes in patient health and ensures timely interventions. 3) Comprehensive medical history access that provides a complete medical history to any healthcare provider involved in the patient's care and prevents oversights and miscommunications. 4) Inter-provider communication that facilitates immediate and secure communication between different healthcare providers and ensures coordinated and informed decision-making.

### Example Use Case

A hypothetical example use of the method and system of the present disclosure is described below. Mr. Patient was seen by Dr. Dentist for multiple teeth extractions. Unfortunately, a communication failure and lack of information led to the death of Mr. Patient shortly after the extractions were performed. Had the disclosed method and system been in place for Mr. Patient, Dr. Dentist would have been alerted to Mr. Patient's extensive medical history, prompting a reassessment of the decision to proceed with the extractions. Additionally, real-time monitoring and alerts could have ensured earlier intervention during Mr. Patient's post-procedure decline, potentially preventing his untimely death. The disclosed method and system represents a groundbreaking advancement in patient safety and healthcare communication. By ensuring that all relevant health data is available and communicated in real-time, the disclosed method and system has the potential to significantly reduce medical errors, improve patient outcomes, and save lives.

At his visit with Dr. Dentist, Mr. Patient indicated on the Medical History Form (16, 17, 18) that he had excessive bleeding following extraction of his teeth in the past. Under medical history he indicated diabetes, heart trouble, and heart attack. Dr. Dentist's chart progress note (13) indicated that the patient's medical history was reviewed and discussed with Mr. Patient and that Mr. Patient denied any current prescription medications (23). Mr. Patient informed Dr. Dentist that he had seen his primary care practitioner (20) within the past 30 days. Mr. Patient indicated that his primary care practitioner had told Mr. Patient that he was “healthy as a horse” and that he no longer required any prescription medications (17, 23, 24).

Dr. Dentist recorded that extraction of all teeth was recommended due to severe disease and manual mobility of all remaining teeth (13, 15, 17, 20, 21, 23, 24, 26). Mr. Patient was clear that he desired immediate treatment. Seeing no contraindications (14, 15, 17, 22, 23), Dr. Dentist performed the extraction of 12 teeth (10, 11, 12, 13). During the procedure, Mr. Patient received 4% Articaine and epinephrine 1:100,000×10 (15, 17, 21, 23, 24, 26). Blood pressure was noted as 124/76 (12, 17). A prescription was sent to the pharmacy (10, 23, 24) and Mr. Patient left the office at 10:00 AM to pick up his prescriptions (21, 23).

Later the same day, Mr. Patient was admitted to a regional emergency department (ED) via emergency medical services (EMS) following an hours-long episode of shortness of breath (1, 2, 3, 7, 16, 17, 20, 23, 24, 26). Per the pre-existing hospital records, Mr. Patient had an extensive medical history including MI, CAD, ischemic cardiomyopathy s/p AICD placement, chronic combined systolic and diastolic congestive heart failure, HTN, diabetes Type I and II, CKD stage 3, above normal BMI, sleep apnea, hyperlipidemia, former smoker, heartburn, and gout (17, 20, 23, 24, 26). None of this was known by any provider from Dr. Dentist through the ED doctor, but was later found by a clerk of the admitting physician. All of this information would be immediately available to the dentist through coordination of (1-13 and 14-27) information as well as the direct provider-to-provider communication portal via the disclosed method and system.

After a wide range of procedures designed to diagnose Mr. Patient, he was admitted to the intensive care unit. Two days later the patient passed away. Mr. Patient's medical trajectory leading up to his untimely death highlights a communication failure between patient and healthcare provider. There appears to have been a critical miscommunication regarding Mr. Patient's overall health status. This misunderstanding likely stemmed from a recent consultation with his primary care practitioner, who reportedly assured him that he was “healthy as a horse” and no longer needed any prescription medications. This optimistic assessment, while perhaps intended to encourage Mr. Patient, seems to have been misconstrued as an all-clear on his chronic and complex medical conditions. Consequently, Mr. Patient might not have fully appreciated the gravity of his health situation, particularly the implications of undergoing multiple teeth extractions without adequate precautions considering his cardiovascular and diabetic history. The choice to proceed with such a significant dental procedure under the belief that he was substantially healthier than he was, resulted in a rapid and severe deterioration in his condition, culminating in his emergency hospital admission and subsequent death.

The incident underscores the importance of ensuring that patients fully understand their health status and the risks associated with medical procedures, especially when chronic conditions are involved. It also calls attention to the need for healthcare providers to verify and clarify patient understandings of their health advice, to prevent dangerous misinterpretations that could lead to life-threatening outcomes. However, with the disclosed method and system, this entire incident could have been averted. The disclosed method and system is able to assess the situation and inform each provider of all data that might impact the health of the patient under any given situation.

The goal of the disclosed method and system is to alert providers at multiple points in the care spectrum. In the scenario above, it is likely that Dr. Dentist would not have performed the procedure if the necessary information was readily available. Even if Dr. Dentist proceeded, there are many potential points prior to the fatal ventricular arrythmia where the disclosed method and system could have alerted providers as to the patient's complete medical situation. Unfortunately, in this case none of the providers had a complete idea of the patient's situation until after his death.

By way of a few examples, the disclosed method and system could have alerted Dr. Dentist of the patient's significant medical history. The patient in this case was not intentionally hiding information from the dentist but misunderstood his situation. Had the disclosed method and system been used in this case, the dentist would have had all information at his fingertips and could have even communicated immediately with the patient's cardiologist for a consult which would likely have halted the procedure. If the dentist proceeded with the procedure, the disclosed method and system would have alerted not only the dentist, but the patient's other providers through information gathered from wearables that the patient's respiratory status had declined shortly after leaving the dentist's office and earlier intervention could have been initiated. The disclosed method and system could have alerted EMS when the patient continued to decline. The first responders would have had immediate access to the providers, the health history, the medication history, and could have directed a more appropriate intervention as opposed to broad spectrum treatments aimed only at the symptoms. Ultimately, if the disclosed method and system had been involved in this case, it is likely that this patient would have survived.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in a different order (or concurrently) without altering the principles of the present disclosure. Unless indicated otherwise, numbering or other labeling of instructions or method steps is done for convenient reference, not to indicate a fixed order. Numerical terms, such as “first,” “second,” and “third,” may be used in the disclosure and claims as unique labels: they are not used to imply a sequence or order unless the context clear indicates otherwise. In other words, a “second element” could be relabeled as a “first element” without departing from the principles of the present disclosure. Further, the presence of a “second element” does not imply or require the presence of a “first element.”

Unless the context clearly indicates otherwise, the singular articles “a,” “an,” and “the” before a noun do not restrict the noun to a single instance. The verbs “comprise,” “include,” and “have” are inclusive and therefore specify the presence of elements without excluding the presence of one or more additional elements.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “coupled,” “engaged,” “adjacent,” “next to,” “on top of,” “above,” “below,” and “disposed.” Unless explicitly described as being “direct,” when a relationship between first and second elements is described in the above disclosure, that relationship encompasses a direct relationship where no other intervening elements are present between the first and second elements as well as an indirect relationship where one or more intervening elements are present (either spatially or functionally) between the first and second elements.

The term “set” generally means a grouping of one or more elements. The elements of a set do not necessarily need to have any characteristics in common or otherwise belong together. However, in various implementations a “set” may, in certain circumstances, be the empty set (in other words, the set has zero elements in those circumstances). As an example, a set of search results resulting from a query may, depending on the query, be the empty set. In contexts where it is not otherwise clear, the term “non-empty set” can be used to explicitly denote exclusion of the empty set—that is, a non-empty set will always have one or more elements.

A “subset” of a first set generally includes some of the elements of the first set. In various implementations, a subset of the first set is not necessarily a proper subset: in certain circumstances, the subset may be coextensive with (equal to) the first set (in other words, the subset may include the same elements as the first set). In contexts where it is not otherwise clear, the term “proper subset” can be used to explicitly denote that a subset of the first set must exclude at least one of the elements of the first set. Further, in various implementations, the term “subset” does not necessarily exclude the empty set. As an example, consider a set of candidates that was selected based on first criteria and a subset of the set of candidates that was selected based on second criteria; if no elements of the set of candidates met the second criteria, the subset may be the empty set. In contexts where it is not otherwise clear, the term “non-empty subset” can be used to explicitly denote exclusion of the empty set.

The phrase “at least one of A, B, and C” should be construed to mean a logical (A OR B OR C), using a non-exclusive logical OR, and should not be construed to mean “at least one of A, at least one of B, and at least one of C.” The phrase “at least one of A, B, or C” should be construed to mean a logical (A OR B OR C), using a non-exclusive logical OR. The phrase “A, B, and/or C” should be construed in the same way as the phrase “at least one of A, B, and C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgments of, the information to element A.

In this application, including the definitions below, the term “module” can be replaced with the term “controller” or the term “circuit.” In this application, the term “controller” can be replaced with the term “module.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); processor hardware (shared, dedicated, or group) that executes code; memory hardware (shared, dedicated, or group) that is coupled with the processor hardware and stores code executed by the processor hardware; other suitable hardware components that provide the described functionality; or a combination of some or all of the above, such as in a system-on-chip.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2020 (also known as the WIFI wireless networking standard) and IEEE Standard 802.3-2018 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

Some or all hardware features of a module may be defined using a language for hardware description, such as IEEE Standard 1364-2005 (commonly called “Verilog”) and IEEE Standard 1076-2008 (commonly called “VHDL”). The hardware description language may be used to manufacture and/or program a hardware circuit. In some implementations, some or all features of a module may be defined by a language, such as IEEE 1666-2005 (commonly called “SystemC”), that encompasses both code, as described below, and hardware description.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The memory hardware may also store data together with or separate from the code. Shared memory hardware encompasses a single memory device that stores some or all code from multiple modules. One example of shared memory hardware may be level 1 cache on or near a microprocessor die, which may store code from multiple modules. Another example of shared memory hardware may be persistent storage, such as a solid state drive (SSD) or magnetic hard disk drive (HDD), which may store code from multiple modules. Group memory hardware encompasses a memory device that, in combination with other memory devices, stores some or all code from one or more modules. One example of group memory hardware is a storage area network (SAN), which may store code of a particular module across multiple physical devices. Another example of group memory hardware is random access memory of each of a set of servers that, in combination, store code of a particular module. The term memory hardware is a subset of the term computer-readable medium.

The apparatuses and methods described in this application may be partially or fully implemented by a special-purpose computer created by configuring a general-purpose computer to execute one or more particular functions embodied in computer programs. Such apparatuses and methods may be described as computerized or computer-implemented apparatuses and methods. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special-purpose computer, device drivers that interact with particular devices of the special-purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

The term non-transitory computer-readable medium does not encompass transitory electrical or electromagnetic signals propagating through a medium (such as on a carrier wave). Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

1. A system comprising:
memory hardware configured to store instructions; and
processor hardware configured to execute instructions stored by the memory hardware, wherein the instructions include:
retrieving a set of protocol data from a first set of data sources;
retrieving a set of user data associated with an individual, wherein the set of user data includes:
historical data associated with the individual from a second set of data sources, and
current data associated with the individual, wherein the current data includes at least one of:
a set of real-time data from a set of sensors monitoring the individual, or
a set of data from a threshold period of time;
determining, based on the set of protocol data, a set of standards;
comparing the set of standards and the set of user data to determine whether the set of standards has been met;
in response to a determination that the set of standards has been met, presenting, via a user interface, an indication with a first formatting; and
in response to a determination that one or more standards of the set of standards has not been met, presenting, via the user interface, a second indication with a second formatting.

2. The system of claim 1, wherein:
the first set of data sources includes at least one of:
a scholarly research database,
a compliance metric database,
a dietary database,
an external alerting system, or
an external guideline repository, and
the set of protocol data includes at least one of:
a clinical guideline,
a regional alert,
a national alert, or
a government agency alert.

3. The system of claim 1, wherein:
the threshold period of time is based on a current status of the individual,
the threshold period of time is based on a current location of the individual, and
the threshold period of time is contiguous with a current time.

4. The system of claim 1, wherein the instructions include:
retrieving a second set of user data associated with a second individual, and
comparing the set of standards, the set of user data, and the second set of user data to determine a priority of the individual and a priority of the second individual.

5. The system of claim 1, wherein the first formatting and the second formatting include at least one of:
a first color and a second color,
a first font and a second font,
a first indicator and a second indicator,
a first text size and a second text size, or
a first alert and a second alert.

6. The system of claim 1, wherein:
retrieving the set of user data is performed in response to a determination that a set of authorization criteria has been met, and
in response to a determination that the set of authorization criteria has not be met, transmitting an authorization request to a device associated with the individual.

7. The system of claim 1, wherein the instructions include:
presenting the historical data in a first manner;
receiving a set of user feedback; and
based on the set of user feedback, presenting the historical data in a second manner.

8. The system of claim 1, wherein the instructions include:
receiving a set of pre-authorization data, and
in response to a determination that a set of authorization criteria has been met, transmitting a data subset of the set of user data to a set of entities, wherein:
the data subset is specified by the set of pre-authorization data,
the set of entities is specified by the set of pre-authorization data,
the set of authorization criteria is specified by the set of pre-authorization data,
the set of authorization criteria includes a status of the individual, and
the set of pre-authorization data is generated based on a set of user inputs.

9. The system of claim 1, wherein determining the set of standards is performed by a machine learning model.

10. The system of claim 1, wherein:
the historical data includes at least one of:
diagnostic records,
treatment records,
surgical notes,
medication logs,
information provided by the individual,
wearable device telemetry,
laboratory reports,
radiology reports, or
family genetic data, and
the second set of data sources includes:
a first healthcare provider; and
a second healthcare provider.

11. The system of claim 1, wherein the system is a distributed computing system that includes:
one or more sets of remote processing hardware; and
one or more sets of remote memory.

12. The system of claim 1, wherein:
the current data includes the set of real-time data from the set of sensors,
the set of sensors is located in a first location, and
the memory hardware and the processor hardware are located at the first location.

13. The system of claim 1, wherein:
the individual is located at a first location, and
the memory hardware and the processor hardware are located in the first location.

14. The system of claim 1, wherein the instructions include:
determining a respective role of a user interacting with the user interface, wherein:
the respective role is one of a set of enumerated roles, and
the set of enumerated roles includes a first role and a second role;
in response to determining that the respective role is the first role, generating the user interface with a first set of elements; and
in response to determining that the respective role is the second role, generating the user interface with a second set of elements.

15. A method comprising:
retrieving a set of protocol data from a first set of data sources;
retrieving a set of user data associated with an individual, wherein the set of user data includes:
historical data associated with the individual from a second set of data sources, and
current data associated with the individual, wherein the current data includes at least one of:
a set of real-time data from a set of sensors monitoring the individual, or
a set of data from a threshold period of time;
determining, based on the set of protocol data, a set of standards;
comparing the set of standards and the set of user data to determine whether the set of standards has been met;
in response to a determination that the set of standards has been met, presenting, via a user interface, an indication with a first formatting; and
in response to a determination that one or more standards of the set of standards has not been met, presenting, via the user interface, a second indication with a second formatting.

16. The method of claim 15, further comprising:
retrieving a second set of user data associated with a second individual, and
comparing the set of standards, the set of user data, and the second set of user data to determine a priority of the individual and a priority of the second individual.

17. The method of claim 15, further comprising:
receiving a set of pre-authorization data, and
in response to a determination that a set of authorization criteria has been met, transmitting a data subset of the set of user data to a set of entities, wherein:
the data subset is specified by the set of pre-authorization data,
the set of entities is specified by the set of pre-authorization data,
the set of authorization criteria is specified by the set of pre-authorization data,
the set of authorization criteria includes a status of the individual, and
the set of pre-authorization data is generated based on a set of user inputs.

18. A non-transitory computer-readable storage medium comprising processor-executable instructions, the instructions including:
retrieving a set of protocol data from a first set of data sources;
retrieving a set of user data associated with an individual, wherein the set of user data includes:
historical data associated with the individual from a second set of data sources, and
current data associated with the individual, wherein the current data includes at least one of:
a set of real-time data from a set of sensors monitoring the individual, or
a set of data from a threshold period of time;
determining, based on the set of protocol data, a set of standards;
comparing the set of standards and the set of user data to determine whether the set of standards has been met;
in response to a determination that the set of standards has been met, presenting, via a user interface, an indication with a first formatting; and
in response to a determination that one or more standards of the set of standards has not been met, presenting, via the user interface, a second indication with a second formatting.

19. The non-transitory computer-readable storage medium of claim 18, wherein the instructions include:
retrieving a second set of user data associated with a second individual, and
comparing the set of standards, the set of user data, and the second set of user data to determine a priority of the individual and a priority of the second individual.

20. The non-transitory computer-readable storage medium of claim 18, wherein the instructions include:
receiving a set of pre-authorization data, and
in response to a determination that a set of authorization criteria has been met, transmitting a data subset of the set of user data to a set of entities, wherein:
the data subset is specified by the set of pre-authorization data,
the set of entities is specified by the set of pre-authorization data,
the set of authorization criteria is specified by the set of pre-authorization data,
the set of authorization criteria includes a status of the individual, and
the set of pre-authorization data is generated based on a set of user inputs.
